(19) United States
(12) Patent Application Publication
(10) Pub. No.: US 2003/0069547 A1
(43) Pub. Date: Apr. 10, 2003

(54) Catheter with retractable perforating or injecting end tool

(76) Inventor: Bertrand Gonon, Lyon (FR)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/266,017

(22) Filed: Oct. 7, 2002

(30) Foreign Application Priority Data
Oct. 9, 2001 (FR) 01 12992

(51) Int. Cl. A61M 5/00

(52) U.S. Cl. 604/263

(57) ABSTRACT

The catheter (1) according to the invention comprises a retractable end tool (9) with a needle (12) in the form of a slide (10) that is integral with a needle base (11) or that pushes this needle under the effect of a continuous or pulsed pressurized fluid. An elastic retraction force, preferably exerted by a spring (21) positioned between the needle-bearing base (11) and the frontal end wall (6) of the catheter, retracts the slide-needle unit to its retracted resting position. This invention is of interest to manufacturers and users of catheters for surgical, therapeutic or diagnostic use.

This invention concerns an improved catheter with a retractable end tool for perforation or injection work.

In the context of a medical treatment, during a surgical or diagnostic procedure, it is often advantageous to administer an active agent, a drug, a product useful for the surgical procedure or a diagnostic product, for example, directly to the diseased area or area to be treated, which is most often situated inside the human body in one of its organs or in its tissues.

Traditionally, this has been accomplished using an injection catheter that is introduced into and advanced along a vessel inside the patient's body until its distal end, pierced with one or more orifices, is as close as possible to the area to be treated. Then the active agent is injected into the area to be treated through the orifice(s) of the end of the injection catheter.

Unfortunately with this traditional device, the agent injected is applied only to the surface of the organ concerned. As a result, it dissipates very quickly and does not permit the deep or long-term treatment of this organ.

For the treatment to be effective, the active product must be applied over a lengthy period or several times, which extends the patient's hospital stay and increases the risk of infection or complications tied to the procedure. Additionally, as the active product is often relatively expensive, the cost of the treatment is increased considerably.

Furthermore, the active product, which is useful and beneficial for the targeted area, may be harmful and even toxic if it is applied to other organs of the patient not involved in the treatment. This is particularly the case with the increasing development of gene therapies during which one can, for example, administer to the target organ genes, cells, nucleic acids or genetically modified proteins applied in isolated fashion or by means of a vector, for example, a virus.

It is therefore very important that the product administered and developed for a specific application remain localized at its injection site. However, with the traditional device described earlier, the active agent is conveyed outside the treatment site by the blood circulation and may thus inopportunely reach other, non-targeted organs.

To remedy this problem, catheters comprising an injection needle device at their distal end have been developed. The needle penetrates into the targeted tissue and the active agent is injected directly through this needle deep within the tissue to be treated.

This makes the active agent much more effective. Indeed, all of the agent injected can act and has much less chance of being unintentionally conveyed toward other non-targeted organs. Furthermore, the tissue is treated deep within it and not just on the surface.

Nevertheless, this type of catheter is very difficult to manipulate for safety reasons. Indeed, placing it in contact with the tissue to be treated, then withdrawing it are very delicate operations due to the presence of the end needle.

To eliminate this disadvantage, injection catheters comprising a retractable needle at their distal end have been proposed in the prior art. In these known devices, the extension of the needle prior to the injection, then the retraction of this needle inside the catheter, are controlled manually by the user.

This manual control of the position of the needle is not yet satisfactory from a safety standpoint. Indeed, if the needle is left in extended position during insertion and positioning of the catheter or when it is retracted, it risks causing serious damage in the patient's body.

There is, therefore, great need for a safer needle injection catheter. One of the objectives of the invention is to respond to this need.

The catheter of the invention is of the injection catheter type, that is, a flexible, guidable tube designed to inject an active product into an organ or into the tissue of an organ. At its distal end, it comprises a retractable perforating or injection tool making it possible to pierce and inject a product directly deep within the tissue of an organ.

The catheter device of the invention ensures greatly enhanced safety by eliminating the risk cited earlier. Indeed, the position of the retractable end tool is controlled automatically; it is extended only when a sufficient amount of pressurized liquid is sent to be delivered by the catheter. If there is no liquid, or if the pressure of the liquid is under a threshold value, the tool is automatically placed in its retracted safety position inside the catheter. Before and after injection, the tool is therefore in retracted position without the user having to intervene. All risk of unintentional damage is thus avoided.

The prior art has also introduced another type of device making it possible to inject and diffuse liquids inside the tissues of an organ of the human body such as the heart, for example. This device, described in patent application WO 00/56232 filed by SAPHIR MEDICAL PRODUCTS GMBH, uses the effect of a pulsed jet of pressurized fluid delivered by a nozzle.

The working of the pressurized jet produces a conduit in the tissue or the organ to be treated, into which an active liquid, growth or other agent is injected or introduced.

Effectively, the working liquid is also the active liquid or a mixture.

The active product sent inside the conduit excavated by the pressurized working liquid is thus administered deep within the tissue to be treated. However, as the end of the conduit is open, the active agent has a tendency to leave the conduit and to be evacuated quickly away from the treatment site. One is then in the same disadvantageous situation noted earlier.

This phenomenon is further aggravated when the organ treated is in motion, which is, for example, the case with the heart. The evacuation of the active product is exacerbated by the metabolic movement of the organ.

Additionally, since the pressurized liquid works longitudinally, the liquid is concentrated at the obstructed end of the conduit. The area of the open end and the walls at the beginning of the conduit receive only a very small amount of the active liquid, even though this end also needs it. The inventive solution described hereinbelow eliminates this disadvantage.

The device of the invention makes it possible to combine the benefits of the pressurized liquid jet with those of needle catheters.

The invention makes it possible to produce the first part of an injection conduit mechanically using a perforating or injecting tool, while diffusing the active product in a layer located close to the surface.

The catheter of the invention has a retractable end tool, preferably a retractable needle that can be used as a discharge nozzle for a pressurized liquid whose work consists in producing a conduit in the tissue layer against which the front wall of the catheter is placed.

Preferably, the active liquid or a portion of this active liquid is also used as working liquid to diffuse the liquid along the length of the conduit. At the end of the pressurized injection or via another quantity sent subsequently with weaker pressure, the active liquid is successfully diffused in the area of tissue situated in the vicinity of the end of the needle, this area not having been or only having been somewhat reached by the active liquid.

The catheter of the invention is shaped like a flexible tube with an end that can be oriented using known means, comprising a retractable perforating or injection end tool that moves between an extended working position and a retracted safety position inside the catheter, allowing the injection of a pressurized liquid.

According to an essential feature of the catheter of the invention, the extension of the end tool is automatic and provoked by the pressure of the liquid escaping from the catheter.

According to another feature, the end tool is automatically retracted inside the catheter if there is no liquid or if the pressure of the liquid is less than a threshold value.

We will describe hereinbelow an embodiment and several variants that must be considered as simple examples.

Other features and advantages of the invention will appear upon reading the following detailed description given in reference to the appended drawings, in which:

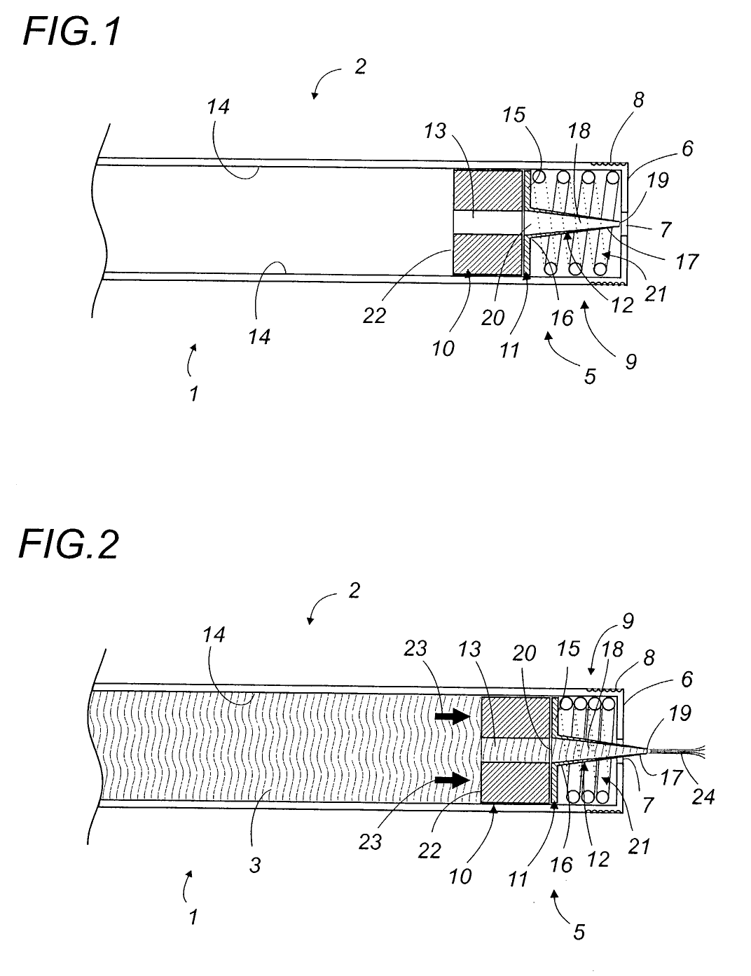

FIG. 1 is a cross section of the end of a mode of embodiment of the catheter of the invention, the needle-nozzle being in retracted resting position;

FIG. 2 is a cross section of the end of an embodiment of the catheter of the invention, the needle-nozzle being in extended liquid injection position;

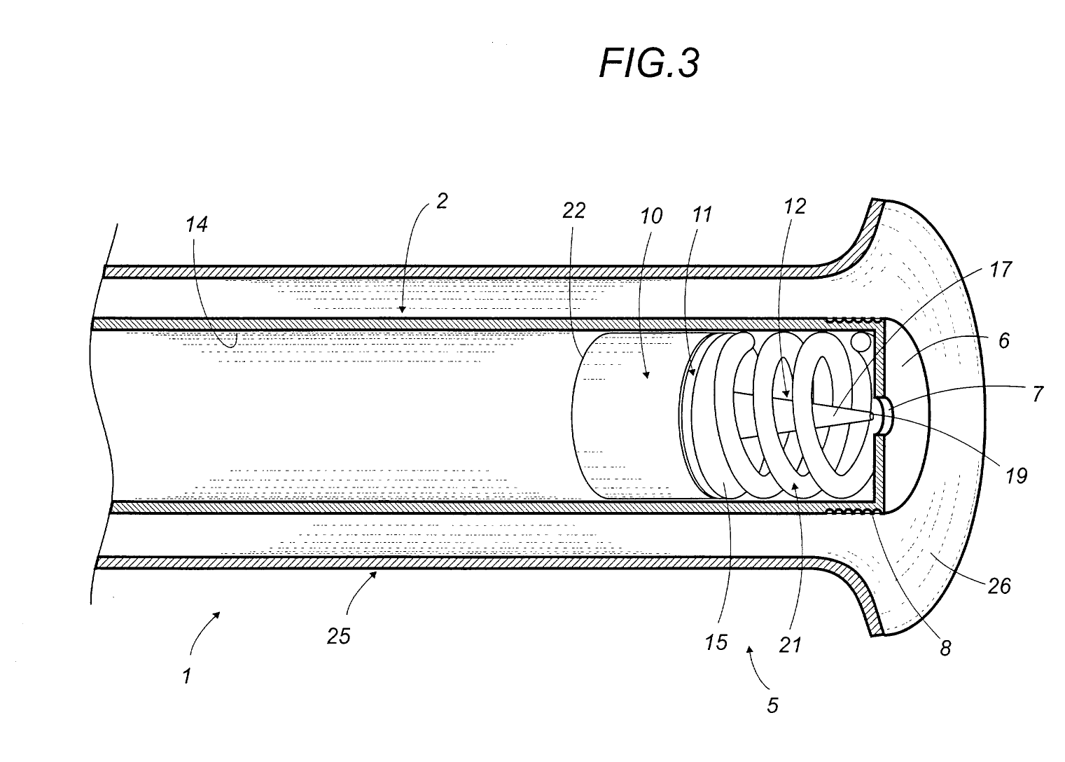

FIG. 3 is a cross section and view in perspective of the end of a variant of the catheter of the invention comprising aspiration means, the needle-nozzle being in retracted position;

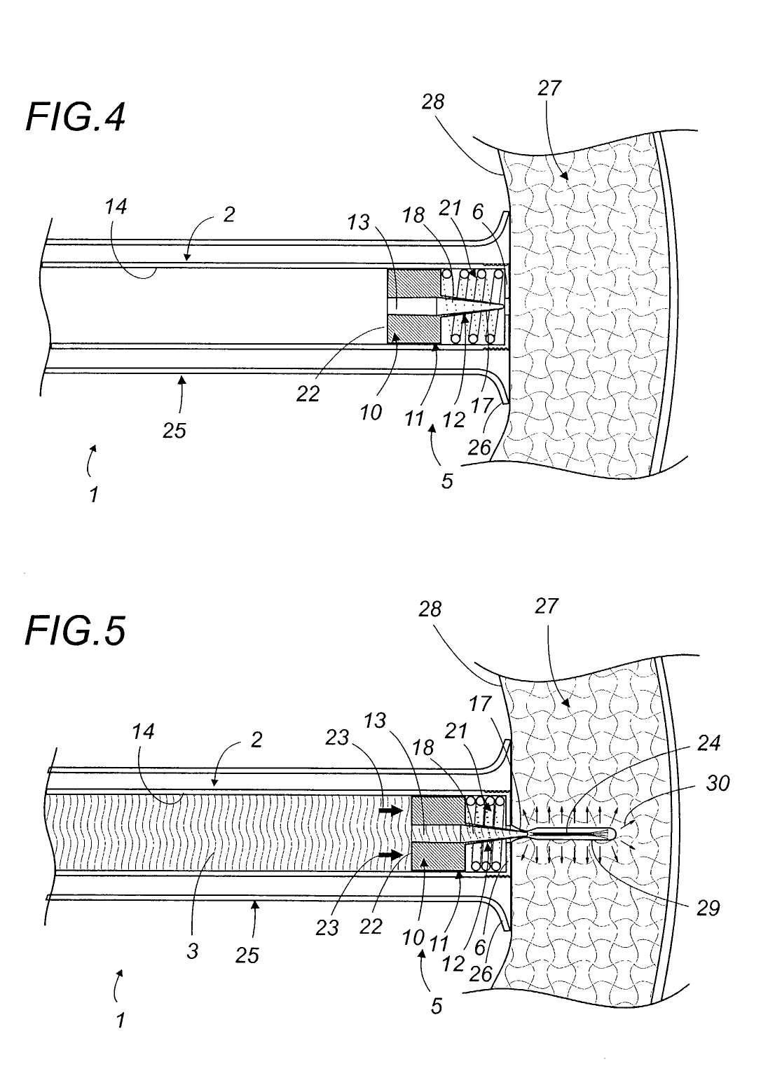

FIG. 4 is a schematic cross section of the end of the aspiration variant of the catheter of the invention positioned in contact with the wall of an organ, the needle-nozzle being in retracted position;

FIG. 5 is a schematic cross section of the end of the aspiration variant of the catheter according to the invention, positioned in contact with the wall of an organ, the needle-nozzle being in extended liquid injection position;

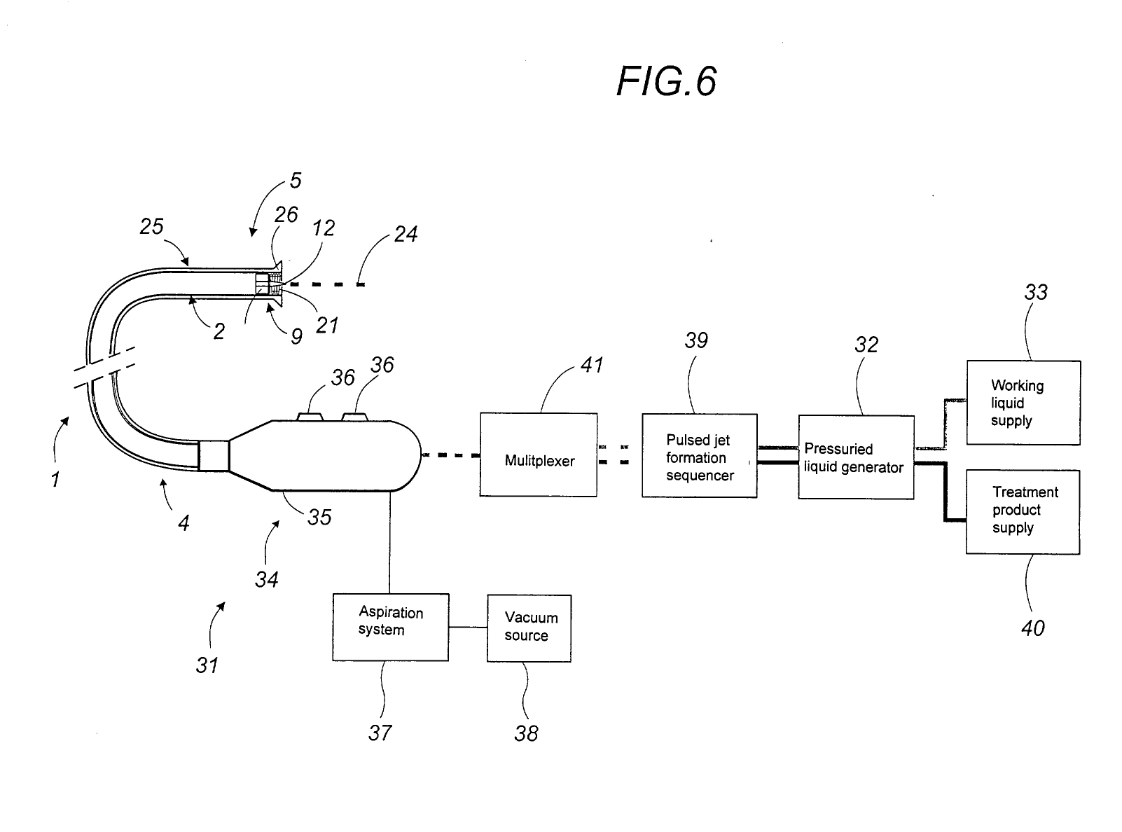

FIG. 6 is a more general explanatory diagram showing a possible application of the catheter of the invention to a pressurized liquid pulsed jet dissection apparatus.

The catheter of the present invention will now be described in detailed fashion in reference to FIGS. 1 to 6. The equivalent elements represented in the different figures will have the same reference numbers.

The catheter (1) according to the invention has the traditional form of a flexible tube (2) in which an active liquid (3) can flow, for example a working liquid, a therapeutic, surgical or diagnostic product.

It comprises a proximal end (4) manipulated by the surgeon and a distal end (5) introduced inside the patient's body.

The distal end (5) comprises known means of navigation and guidance that are not shown in the figures, for they are not part of the invention.

The catheter terminates at its distal end in a front end wall (6) pierced by an orifice (7), preferably central, through which the active liquid (3) can flow or be ejected.

Before the surgical procedure, this end (5) of the catheter may be protected by a removable cover, for example a screw cap. In this case, it comprises external threading (8).

The distal end of the catheter according to the invention houses a retractable end tool (9) that moves between a retracted safety position inside the catheter, shown in FIGS. 1, 3, 4 and 6, and an extended working position, shown in FIGS. 2 and 5.

The end tool (9) makes it possible to perform perforation or injection work.

According to a preferred embodiment, it comprises a slide (10) and a base (11) bearing a needle-nozzle (12).

The slide (10) is preferably shaped like a roughly cylindrical part that is hollow or pierced by a through channel (13).

Its external diameter is slightly smaller than the internal diameter of the tube (2) so that it can slide inside this channel. However, the play between the tube (2) and the slide (10) remains minimal in order to ensure its leakproofness despite this sliding freedom.

Preferably, the internal wall (14) of the tube (2) and the parts of the slide in contact with it are coated with a Teflon-based coating ensuring its leakproofness even at high pressure, while fostering the sliding action.

Likewise, all the sliding parts of the end tool (9) in contact with the internal wall (14) of the tube (2) are preferably coated with a Teflon-based coating.

The slide (10) is integral with or capable of pushing the needle base (11) in order to drive its movement represented in FIGS. 4 and 5. The slide (10) and the base (11) may be produced from a single part.

The needle base (11) comprises a mounting plate (15) that is preferably roughly circular and whose diameter is smaller than the internal diameter of the tube (2) so that it can slide inside this tube. The needle (12) projects from this backing plate.

Preferably, the parts of the needle-bearing base (11) in contact with the internal wall (14) of the tube (2) are also coated with a Teflon-based coating.

The needle (12) is connected to the mounting plate (15) by a base (16) and ends in a tip (17) capable of performing the perforation or injection work. It is traversed by an internal channel (18) that opens at the end of the tip (17) via an ejection orifice (19).

Preferably, the internal channel (18) of the needle has a roughly conical shape that narrows toward the ejection orifice (19) in order to act as a nozzle.

In the embodiments represented, the needle (12) also has an approximately conical external shape. However, this shape is not limiting. Indeed, the length and the shape of the needle (12) may be optimized depending on the application envisioned for the catheter of the invention. Thus, the needle (12) may, for example, be pointed, rounded or beveled.

Additionally, the external surface of the needle is not necessarily smooth. It may comprise raised areas, such as, for example, one or more twists, notches, hooks or retaining teeth in order to improve the anchoring and the engagement of the needle in the tissue.

The mounting plate (15) is also pierced by a through channel (20) whose ends are in fluid communication for one with the end of the channel (13) of the slide (10) and for the other with internal channel (18) of the needle (12).

The tip (17) of the needle (12) is situated opposite the orifice (7) of the front end wall (6) of the catheter in order to be able to extend outside the catheter through this orifice when the needle (12) is in extended working position (FIGS. 2 and 5).

When the needle (12) is in retracted safety position (FIGS. 1, 3, 4 and 6), a liquid passing through the internal channel (18) of the needle can also flow outside the catheter through this orifice (7).

According to an essential feature of the device according to the invention, an elastic retraction force retracts the slide-needle unit into retracted resting position, that is, into retracted safety position inside the catheter.

This retraction force may be exerted by a spring (21) preferably positioned between the base plate (15) of the needle-bearing base (11) and the frontal end wall (6) of the catheter.

The operation of the device according to the invention is obvious from the preceding description.

If there is no fluid in the tube (2) (FIGS. 1, 3 and 4), the spring (21) exerts an elastic retraction force on the needle-bearing base (11), which keeps the needle (12) in retracted resting position.

When fluid is sent under pressure into the tube (2) toward the distal end (5) of the catheter, it pushes on the rear frontal face (22) of the slide (10). The pressure exerted by the fluid has been represented in FIGS. 2 and 5 by black arrows (23) drawn with thick lines.

If the pressure of the fluid is sufficient to offset the elastic retraction force of the spring, the slide (10) slides inside the tube (2) toward the distal end (5) of the catheter, drawing with it the needle-bearing base (11), and compressing the retraction spring (21).

The movement of the base (11) causes the tip (17) of the needle (12) to extend outside the catheter through the orifice (7) of the frontal end wall (6). The needle (12) is then in extended working position (FIGS. 2 and 5).

The fluid is ejected from the catheter (1) through the orifice (19) of the needle-nozzle (12) in the form of a jet (24).

When the pressure of the fluid decreases at the end of injection, the retraction spring (21) decompresses and pushes the back plate (15), which causes the needle-bearing base (11) and the slide (10) to slide toward the proximal end (4) of the catheter. The needle (12) then resumes its retracted resting position.

It should be stressed that in particularly advantageous fashion in the device according to the invention, the extension and retraction of the needle is accomplished automatically and without direct intervention on the part of the user, based on the pressure of the fluid flowing in the catheter.

The surgeon only controls the starting and stopping of the flow of liquid, the position of the needle being automatically and virtually immediately adapted to the situation.

To cause the needle (12) to extend, the fluid must have sufficient pressure to succeed in compressing the spring (21). There is, therefore, a limit or threshold pressure for the fluid, corresponding to the equilibrium pressure with the elastic retraction force, below which the needle will not extend. This threshold pressure is fixed in this embodiment by the construction and depends on the rigidity constant of the retraction spring (21).

It is preferably in the neighborhood of 5 bars.

Advantageously, it is thus possible to deliver a low-pressure liquid without the needle extending. One can thus; more particularly, purge the catheter with a low-pressure physiological serum, for example at 3 bars of pressure, or use the catheter of the invention for a washing function.

According to the procedures envisioned, it is often particularly advantageous to deliver a pulsed jet of pressurized liquid. The catheter according to the invention makes it possible to achieve this objective in satisfactory fashion.

Indeed, even though the needle retracts automatically when the pressure of the liquid decreases, this occurs with a slight delay. As a result, if the pulses of the pulsed jet are sufficiently close to one another, the needle does not have time to retract between the different pulses of a same blast. Thus, the needle extends upon the first pulse and does not retract until after the last pulse of a same pulsed blast of pressurized liquid.

The catheter of the present invention may also comprise an aspiration mechanism opening close to the intervention area, which allows the aspiration of the working liquid, body fluids and small debris, thereby improving the effectiveness of the procedure and the visibility of the area treated for the surgeon.

A variant of this type has been represented in FIGS. 3 to 6. The catheter (1) comprises an aspiration conduit (25), for example generally cylindrical in shape and surrounding the conduit (2), preferably concentrically.

Preferably, the aspiration conduit (25) flares at the distal end (5) of the catheter until it forms a skirt (26).

Although the penetration of the needle (12) into the tissue treated offers a certain stability at the distal end of the catheter during injection, the catheter of the invention may comprise an additional anchoring system in order to ensure the immobilization of its active end when pressurized liquid is sprayed, thereby improving the accuracy of the procedures.

This may involve any mechanical anchoring means or a means using the aspiration of the catheter and, for example, the skirt (26).

An example of the use of the catheter according to the invention has been schematized in FIGS. 4 and 5.

The surgeon introduces the distal end of the catheter inside the patient's body and guides it along the appropriate vessel until it arrives near an organ or tissue (27) to be treated.

After having identified, using identification means not shown, an area of the wall (28) appropriate for the treatment, he positions the frontal end wall (6) of the catheter as close as possible to the targeted area of the wall (28) using orientation and articulation means that are also not shown.

He then starts the aspiration in the aspiration conduit (25). The portion of wall (28) of the tissue (27) located opposite the inside of the skirt (26) is then aspirated and presses against the frontal end wall (6) of the catheter. The distal end of the catheter is thus immobilized in spraying position as illustrated in FIG. 4.

The surgeon then triggers the jet of pressurized working fluid, which, when it reaches the end of the catheter, automatically causes the needle (12) to extend through the orifice (7) of the frontal wall (6) of the catheter. The tip (17) of the needle passes through the wall (28) and penetrates into the tissue (27).

The maximum depth of penetration into the tissue (27) depends on the maximum length of travel of the slide (10) and therefore on the possible compression of the spring (21). It is preferably around 2 mm.

The pressurized liquid passes through the channel (13) of the slide (10), the channel (20) of the base (11) and the channel (18) of the needle (12), which plays the role of nozzle. It is then ejected at high speed through the orifice (19) of the needle into the tissue (27) where it excavates a conduit (29).

With the catheter according to the invention, it is the needle (12) that, through its mechanical penetration, begins the work of excavating the conduit (29). This advantageous feature makes it possible to reduce the pressure of the working liquid and/or the number of blasts necessary to produce the conduit (29).

This feature is particularly interesting when the organ targeted is surrounded by a membrane or an external wall that is more resistant than its internal tissue. The needle (12) passes through the external wall, and the pressurized liquid can then easily excavate the more tender internal tissue.

The surgeon can then trigger the injection of an active, treatment, growth, diagnostic or other agent into the conduit (29).

In effective fashion, the working liquid is also the active liquid or a mixture containing the active agent.

The active product sent inside the conduit (29) excavated by the pressurized working liquid is then administered deep within the tissue to be treated.

It diffuses through the walls of the conduit (29) and penetrates the tissues adjacent to it, which act as a sponge. These tissues do not suffer any damage, for the pressure of the fluid is very low perpendicular to the longitudinal direction of the conduit. The active liquid thus penetrates all the venulae and arteriolae passing through the conduit. The diffusion of the active product, represented by the thin arrows (30) in FIG. 5, is accomplished through the walls of the conduit (29) in all directions.

At the end of the injection, the pressure of the liquid decreases, and the needle resumes its resting position inside the catheter. The surgeon stops the suction to remove the end of the catheter from the wall (28) of the tissue. He can then withdraw the catheter from the patient's body in complete safety, since the needle is in retracted position.

One possible application of the catheter of the invention to a dissection apparatus (31) that operates via pulsed jets of pressurized liquid has been represented schematically in FIG. 6.

The dissection apparatus (31) makes it possible to send one or more jets (24) of pressurized sterile liquid, for example, against a tissue to be dissected or matter to be disaggregated.

This apparatus comprises a pressurized liquid generator (32) connected to a supply (33) of working liquid. The pressure of the generated liquid jet can be adjusted in order to adapt it to the needs.

The working liquid used is preferably a sterile physiological serum. But other sterile liquids may obviously be used as working fluid like, for example, a saline solution, a glucose solution, a Ringer's lactate solution, a hydroxyethyl starch solution or a mixture of these solutions.

The sterile working liquid is conveyed to a hand unit (34) allowing the surgeon performing the procedure to control the start of the working liquid jet and to direct it. The hand unit (34) comprises an ergonomic body (35) enabling easy handling and holding, and presenting control mechanism such as push buttons, for example.

The hand unit (34) is extended by the catheter (1) of the invention delivering the jet of pressurized sterile liquid in order to perform surgical cutting, dissection or disaggregation work.

The surgical apparatus (31) preferably comprises an aspiration system (37) connected to a vacuum source (38), for example the hospital's general vacuum circuit.

The aspiration system ends in an aspiration conduit (25) concentric to the tube (2) and opening near the skirt (26).

For improved efficacy, the surgical apparatus (31) is preferably a pulsed jet apparatus that sends the pressurized liquid via firing in the form of a discontinuous pulse train of simple jets of pressurized liquid. To do this, the surgical apparatus (31) comprises a sequencer (39) allowing the formation of the pulsed jet and controlling its parameters.

In order to allow the application of a treatment product, the dissection apparatus also comprises a supply (40) of treatment product.

The treatment product is preferably in liquid form or sufficiently fluid to be applied using the surgical apparatus (31). This may be a single active agent or a mixture of several active agents in fluid, for example, and preferably in liquid form, pure or in solution or suspension in any solvent or in the form of an emulsion, a foam or a gel.

In FIG. 6, the working liquid has been symbolized by a thick black line and the treatment fluid by a thick gray line.

The dissection apparatus may, for example, comprise, upstream of the catheter (1), a switching mechanism allowing the surgeon performing the procedure to fire a jet of working liquid or a jet of treatment product.

The apparatus may also comprise, in place of the switching mechanism, a mixing mechanism allowing the surgeon to produce a mixture of working fluid and treatment product at the appropriate time.

When the surgical apparatus (31) delivers a pulsed jet of pressurized liquid, it may also comprise a multiplexer (41) making it possible to combine pulses of working liquid and of treatment product within the discontinuous pulse train constituting the pulsed jet. The parameters of the different pulses, their type and succession may be modified and programmed according to the needs of the surgeon, the patient and the type of procedure performed, in order to optimize the result of the procedure.

The catheter according to the invention is obviously not limited to an application to one surgical device as described above. It is possible to imagine numerous other surgical, therapeutic or diagnostic apparatuses to which it can be adapted.

The catheter according to the invention is particularly suited to performing transmyocardial or myocardial revascularization procedures consisting of excavating a number of revascularization conduits in an ischemic area of the wall of the myocardium.

However, its use is not limited to a cardiac procedure. It is possible to imagine many applications of the catheter according to the invention for procedures on all types of organs or tissue of the human or animal body.

1. Catheter composed of a flexible, guidable tube (2) and comprising a retractable perforation or injection end tool that moves between an extended working position and a retracted safety position inside the catheter, allowing the injection of a pressurized liquid, wherein it comprises a mechanism (10) that moves under the effect of the pressure of the liquid (3) escaping from the catheter (1), which causes the automatic extension of the end tool (9).

2. Catheter according to claim 1, wherein it comprises a means of retraction (21) causing the automatic withdrawal of the end tool (9) inside the catheter in the absence of any liquid (3) or if the pressure of the liquid is lower than a threshold value.

3. Catheter according to the preceding claim, wherein the threshold value for the pressure of the liquid (3) is in the neighborhood of 5 bars.

4. Catheter according to any of the preceding claims, wherein the end tool (9) includes a retractable needle (12).

5. Catheter according to the preceding claim wherein the needle (12) plays the role of discharge nozzle for the pressurized liquid (3).

6. Catheter according to claim 4 or 5, wherein the needle (12) is approximately conical, pointed, rounded or beveled.

7. Catheter according to any one of claims 4 through 6, wherein the external surface of the needle (12) has raised areas.

8. Catheter according to any one of claims 4 through 7, wherein the end tool (9) includes a slide (10) and a base (11) bearing a needle (12).

9. Catheter according to the preceding claim, wherein the slide (10) is a roughly cylindrical part whose external diameter is slightly smaller than the internal diameter of the tube (2) and is pierced by a through channel (13).

10. Catheter according to claim 8 or 9, wherein the slide (10) can slide in the tube (2) of the catheter and is integral with or capable of pushing the needle-bearing base (11) in order to drive it in its sliding motion.

11. Catheter according to any one of claims 8 through 10, wherein the needle-bearing base (11) comprises a mounting plate (15) that is roughly circular and whose diameter is smaller than the internal diameter of the tube (2), from which the needle projects (12).

12. Catheter according to the preceding claim, wherein the mounting plate (15) is pierced by a through channel (20) whose ends are in liquid communication for one from the end of the channel (13) of the slide (10) and second with the internal channel (18) of the needle (12).

13. Catheter according to any one of claims 8 through 12, wherein the slide (10) and the base (11) are made from one piece.

14. Catheter according to any one of the preceding claims, wherein the internal wall (14) of the tube (2) of the catheter and the sliding parts of the end tool (9) in contact with them are coated with a Teflon-based coating.

15. Catheter according to any one of the preceding claims, wherein an elastic retraction force retracts the end tool (9) to its retracted position.

16. Catheter according to the preceding claim, wherein the retraction force is exerted by a spring (21).

17. Catheter according to the preceding claim, wherein the spring (21) is positioned between the mounting plate (15) of the needle-bearing base (11) and the frontal end wall (6) of the catheter.

18. Catheter according to any one of the preceding claims, characterized in that it also comprises an aspiration mechanism opening near the intervention area.

19. Catheter according to the preceding claim, wherein the aspiration mechanism comprises an aspiration conduit (25) that is generally cylindrical in shape, surrounding the conduit (2) concentrically and flaring at the distal end (5) of the catheter until it forms a skirt (26).

20. Catheter according to any one of the preceding claims, wherein it also comprises an anchoring mechanism making it possible to ensure the immobilization of its active end (5) during releases of pressurized liquid.

21. Catheter according to claims 18 and 20, wherein the anchoring mechanism uses the aspiration of the catheter.

22. Catheter according to any one of the preceding claims, wherein the maximum depth of penetration of the end tool (12) in a tissue (27) is around 2 mm.

23. Catheter according to any one of the preceding claims, wherein its distal end (5) can be protected by a removable cover.
